(19) United States
(12) Patent Application Publication
(10) Pub. No.: US 2019/0021988 A1
(43) Pub. Date: Jan. 24, 2019

(54) ENHANCED TRANSDERMAL DELIVERY OF ACTIVE AGENTS

(71) Applicant: Bruce J. SAND, Westlake Village, CA (US)

(72) Inventor: Bruce J. SAND, Westlake Village, CA (US)

(21) Appl. No.: 16/070,494

(22) PCT Filed: Jan. 23, 2017
(86) PCT No.: PCT/US2017/014621

§ 371 Date: Jul. 16, 2018

Related U.S. Application Data
(60) Provisional application No. 62/388,310, filed on Jan. 23, 2016.
(60) Provisional application No. 62/390,250, filed on Mar. 23, 2016.

(51) Int. Cl. A61K 9/00; A61K 47/10; A61K 47/24; A61K 47/14; A61K 47/22; A61K 47/42; A61K 9/107

(52) U.S. Cl. CPC A61K 9/0014; A61K 47/10; A61K 47/24; A61K 9/1075; A61K 47/22; A61K 47/42; A61K 47/14

(57) ABSTRACT

Improved formulations that combine chemical permeation enhancers with additional agents so that the formulations simultaneously penetrate both lipid and cellular matrices provide effective transdermal delivery of active agents.

### CROSS-REFERENCE TO RELATED APPLICATIONS

This application claims priority from provisional application U.S. Ser. No. 62/388,310 filed 23 Jan. 2016 and from provisional application U.S. Ser. No. 62/390,250 filed 23 Mar. 2016. The contents of these documents are incorporated herein by reference in their entirety.

### TECHNICAL FIELD

The invention is in the field of enhanced transdermal delivery of active agents via disruption of the structural cellular and lipid components of the stratum corneum.

### BACKGROUND ART

Transdermal drug delivery is an attractive route of administration, whereby the drug is delivered via the skin for local or systemic distribution. Transdermal delivery of drugs and other active agents is noninvasive and has the potential for the controlled release of drugs while avoiding the significant first-pass effect of drugs through the liver, associated poor bioavailability and frequent painful hypodermic injection.

The stratum corneum (SC), the outermost layer of the epidermis is, however, a formidable permeation barrier for topically administered agents. In fact, it is generally believed that the skin only permits the permeation of small and lipophilic drugs of low molecular weight (less than 500 Da). Nevertheless, in view of its theoretical advantages, enormous efforts have been expended on the development of new approaches to enhance transdermal drug delivery.

The SC presents a unique structural heterogeneity, that of a “bricks and mortar organization” with cells, the corneocytes, serving as the “bricks” with the “mortar”, in the form of a lipid milieu, sequestered within the extracellular spaces, where it is organized into lamellar bilayers that surround the corneocytes.

Hence, penetrants designed to convey active agents through the skin may do so by penetrating the lipid bilayers or penetrating the corneocytes or both. Penetration of the lipid milieu is believed to be enhanced by formation of micelles that self-assemble by virtue of inclusion of amphiphilic carriers in the penetrant. Micelle formation is enhanced by equimolar formulations of amphiphilic polymers as well as by milling. Milling, generally, alters the shape of the micelles to make them more effective. It has also been shown that micellular stability is enhanced by inclusion of an electrolyte. Typical formulations designed to enhance transport through the lipid milieu thus include amphiphilic polymers such as those that make up lecithin organogels as described in PCT publication number WO2016/105499. As described in said publication, small amphiphilic molecules such as benzyl alcohol also enhance the effectiveness of the formulation.

Human SC typically comprises about 20 corneocyte cell layers. The cellular interior is comprised of tightly packed keratin filaments. Keratin, a fibrous protein, is the most abundant protein in the skin. Keratins belong to the superfamily of intermediate filament proteins and consist of long polypeptide chains stabilized by disulfide bonds, which are tightly packed either in α-chains (α-keratins) or in β-sheets (β-keratins). These filaments impart mechanical strength to the corneocyte, without which the cell becomes fragile and prone to rupturing upon physical stress.

The high-degree of cross-linking by the disulfide bonds, hydrophobic interactions and hydrogen bonds between the keratin filament structures within the individual corneocytes confer its mechanical stability. Therefore, keratinous material is water insoluble and resistant to degradation by proteolytic enzymes, such as trypsin, pepsin and papain.

Yet despite the clear importance of these corneocytes both as spacers and as a scaffold for the extracellular lipid matrix, transdermal drug delivery has been primarily focused upon disruption of the extracellular lipid milieu. It has been traditionally assumed that the extra-cellular, lipid enriched matrix of the SC comprises the primary structure that limits transdermal delivery of hydrophilic drugs. This may not, in fact, be completely accurate. Recent studies suggest that the cellular component also plays a significant role in the barrier function of the SC, that derives from a highly packed layer of terminally differentiated corneocytes.

Skin's electrical resistance or impedance is generally considered a marker of skin permeability and changes in skin resistance due to exposure to different penetrants has been shown to correlate with increased skin permeability to model drug compounds. From a mechanistic viewpoint, skin's electrical resistance is known to be governed primarily to the highest ordered, lipophilic barrier of the SC lipid bilayers. Therefore, changes in skin's resistance are a sensitive measure of changes in the SC lipid bilayer integrity. Changes in skin's resistance are seen to occur with a lag time of one or more hours, which suggests a kinetic barrier that may be a diffusive transport limitation. Measurement of skin's resistance or impedance can be used to as a ‘generic’ measurement of skin permeability that does not depend on the specific characteristics of target molecules, such as hydrophobicity and charge.

In general, modes of entry through the skin are summarized in FIG. 1. Any of these may be employed by the invention formulations.

An additional penetrant for delivering an active agent through the skin, in addition to or in conjunction with the penetrants specifically designed to convey agents through the lipid matrix and/or through the corneocytes, may operate in unknown mechanisms as exemplified by a class of peptides generally termed “skin penetrating peptides” (SPPs). These may also be cell penetrating peptides (CPPs). Documents describing these SPPs are cited hereinbelow. SPPs have been shown to enhance delivery of macromolecules, such as genetic material (DNA, etc.), botulinum neurotoxin, human growth hormone, insulin, etc. SPPs drive skin penetration via co-administration or fusion without interaction with or modification of the guest active agent and are considered peptide-chaperones.

The mechanism of penetration provided by SPPs is unclear. However, SPP treatment has been demonstrated to result in a statistically significant increase in percentage of a-helices of keratins, suggesting that SPPs may stabilize these structural proteins in the skin rather than denaturing them. SPPs bind to keratin proteins through hydrogen bonds and weak electrostatic interactions and thus operate as binding mediators between keratin and drug molecules. It has thus been assumed that SPPs function by increasing partitioning into keratin-rich corneocytes due to their affinity towards keratin, thus avoiding the lipid milieu.

SPPs may also utilize pathways between corneocytes via diffusion of drug via gaps between cells as well as through lipid bilayers, but without disruption. One typical SPP, TD-1, is known to loosen the desmosome-induced tight junctions between corneocytes with a change in the space between cells from about 30 nm to about 466 nm in 30 minutes from topical application. The cell gaps increase and then gradually are restored in 1 hour after treatment with TD-1.

In contrast to SPPs, traditional chemical permeation enhancing formulations (CPEs), rely upon disrupting the extra-cellular lipid matrix with resultant increased transepidermal water loss (TEWL) and decreased skin electrical resistance, but have been, for the most part, ineffective in delivering macromolecules.

The penetrants that are the subject of the present invention take advantage of the various effects of the foregoing types of penetration enhancers to provide effective penetration vehicles for a desired active agent.

All documents cited herein are hereby incorporated by reference.

### DISCLOSURE OF THE INVENTION

This invention employs combinations of components that target the barriers presented both by the extracellular lipid milieu, as well as by the cellular (corneocyte) components, and in some embodiments, the mechanism of penetration accessed by SPPs. In some embodiments, the self-assembly of copolymers into micelles is employed to aid penetration.

The invention provides two major embodiments. In one embodiment, an improved composition designed basically to permeate the protective lipid layers is employed. This improved composition may also be supplemented with components that act in alternative ways to achieve penetration of the skin, including disruption of the corneocytes themselves and the use of skin penetrating peptides (SPPs) and other permeation-enhancing agents to act in a synergistic manner with the basic composition. A second embodiment employs a known penetration vehicle but supplements this vehicle with these additional complementing components.

In one aspect, the invention is directed to a vehicle for effecting transdermal penetration of an active ingredient wherein said vehicle comprises: an approximately 1:1:1 equimolar mixture of bile salt:lecithin:completion component; one or more electrolytes; one or more surfactants; and benzyl alcohol or an analog thereof. In some embodiments, the vehicle also includes at least one SPP and/or a keratinolytic agent and/or a permeation enhancer.

In a second aspect, the invention is directed to a vehicle for effecting transdermal penetration of an active ingredient wherein said vehicle comprises: lecithin organogel; benzyl alcohol or an analog thereof; and keratinolytic agent. In one embodiment, the vehicle comprises 25-70% w/w lecithin organogel and 0.5-20% w/w benzyl alcohol or an analog thereof.

In a third aspect, the invention is directed to a vehicle for effecting transdermal penetration of an active ingredient wherein said vehicle comprises: lecithin organogel; benzyl alcohol or an analog thereof; and at least one SPP. In one embodiment, the vehicle comprises 25-70% w/w lecithin organogel and 0.5-20% w/w benzyl alcohol or an analog thereof.

These second and third aspects may be combined and/or further include a permeation enhancer.

In general, the present invention embodies chemical permeation enhancement methods and formulations (CPEs), which are believed to be largely directed to the selective disruption of both the extracellular lipid matrix and/or the intracellular milieu of the SC. These topical formulations are designed to host various guest molecules, deliver them expeditiously across the SC barrier, prevent the premature release of the drug cargo, transport them to their target sites and render them bioavailable.

Thus various synergistic combinations of (1) at least binary CPE mixtures, (2) biosurfactant-based reverse wormlike-micellar systems, (3) bipolar aliphatic alcoholic solvents, (4) corneocyte-degrading keratinases, (5) thiol-moiety reducing agents, and (6) skin penetrating peptides (SPPs) are included in the invention and (7) permeation enhancers.

In addition, the invention is directed to formulations that include active components to be administered to a subject in a transdermal manner wherein transport is made effective by the vehicles of the invention as well as to methods to administer these compositions or formulations by applying them to the skin or nails of an appropriate subject. Thus, methods to administer antibodies, nutritional supplements, drugs, diagnostic agents, and the like are included in the invention.

### BRIEF DESCRIPTION OF THE DRAWINGS

FIG. 1 shows the pathways into the skin for transdermal drug delivery. “A” is transdermal transport via within extracellular lipids; “B” is transport through hair follicles and sweat ducts; “C” is transport directly across the SC; and “D” is stripping, ablation and microneedles produce larger pathways across the SC.

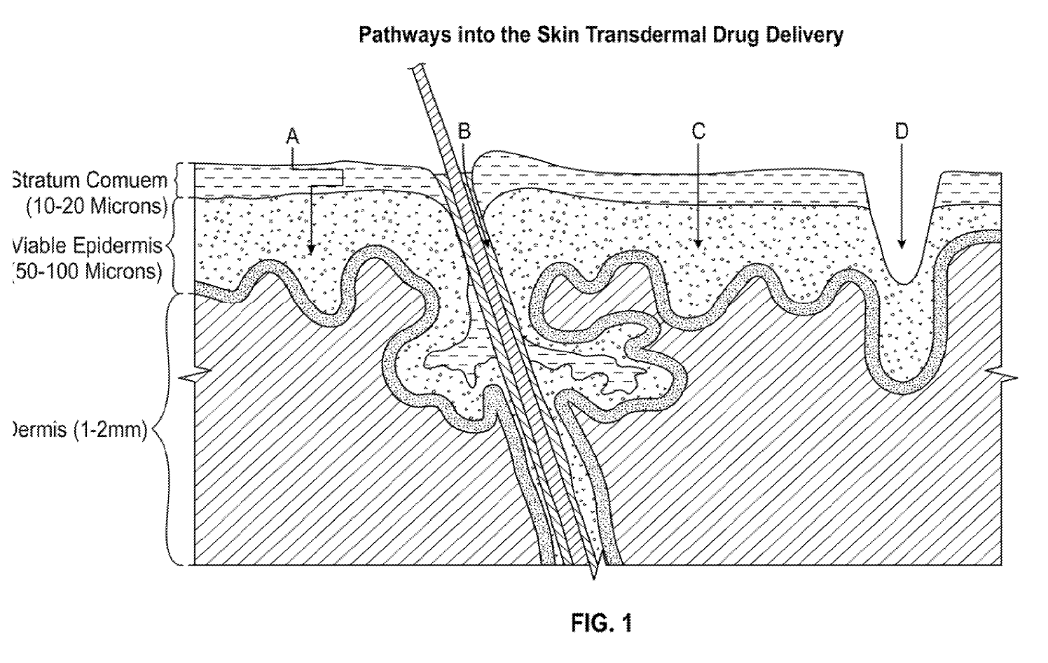

FIG. 2 shows the effect of solvent on micelle formation.

FIG. 3 is a schematic of the reverse micellar structures formed by lecithin with and without bile salt.

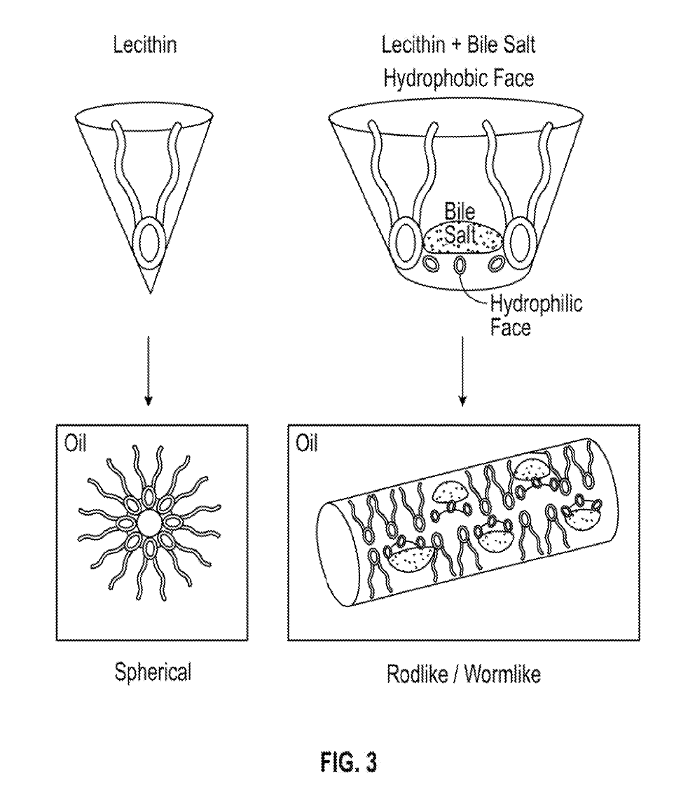

### MODES OF CARRYING OUT THE INVENTION

The invention is directed to vehicles that are useful in carrying active ingredients through the dermis of a subject either to reside locally in a subdermal area or systemically. The subjects are typically human, but the vehicles are useful for administration to any subject that is protected by a dermal layer. Such subjects include various animal subjects including mammals, birds, reptiles, fish and any other creature that is protected by a lipid matrix supporting corneocytes that comprise keratin networks. The active agent may be a therapeutic, a diagnostic, a nutrient or any other agent that needs to cross the dermal barrier.

In general, the vehicles of the invention are useful in the transport of any type of active agent, although certain embodiments may be preferred depending on, for example, the molecular weight and/or hydrophilicity and hydrophobicity of the active agent. For example, inclusion of SPPs is particularly advantageous in the transport of macromolecules such as proteins and oligonucleotides whereas the improved chemical permeation penetration enhancers (CPEs) are sufficient for the transport of small molecules such as lidocaine or nutrients such as amino acids. In general, the selection of the appropriate vehicle for the active agent to be administered and for the subject for whom the active agent is intended is well within the skill of the ordinary artisan.

Thus, the present invention provides improved skin penetrating compositions that may be employed to transport drugs and/or diagnostics through the skin barrier and into a subdermal local location and/or into systemic circulation for a variety of subjects and active agents.

In general, penetration is improved when the formulation comprises micelles. The formulations of the invention may self-assemble into micelles, in particular micelles with a wormlike shape. While lecithin alone forms vesicles or micelles, these micelles are inherently unstable because the bulky hydrophobic tails of the lipid (lecithin) inhibit its solubility in water and may release their cargo of active agents prematurely. The addition of second class of biosurfactants, bile salts, even in small amounts will intercalate into lecithin vesicles and stabilize these structures.

In addition, modified lecithin microemulsion-based organogels are thermodynamically stable, clear, viscoelastic, biocompatible and isotropic phospholipid structured systems. The naturally occurring surfactant, lecithin, can form reverse micelle-based microemulsions in non-polar environment because of its geometric discipline. These small reverse micelles upon addition of a specific amount of water, likely grow monodimensionally into long flexible and cylindrical giant micelles, above a critical concentration of lecithin. These giant micelles form a continuous network that immobilizes the external organic phase forming a gel or jelly-like state.

Formation of wormlike micelles is also enhanced by a background electrolyte at sufficient levels. These electrolytes, such as sodium citrate, are required to more effectively increase viscosity and viscoelasticity of micelles and screen the repulsion between bile salt anions at a minimal concentration. Another effect of sodium citrate is its ability to “salt out” solutes from water as the Hofmeister effect. In other words, a specific molar ratio and a sufficient electrolyte concentration are helpful for the formation of stable, long flexible cylindrical micelles. One favorable molar ratio of bile salt to lecithin is 1:1, but the concentration of electrolyte is determined by titration of the solution to transparency of the solution and enhanced viscosity as determined when the solution container is inverted.

In embodiments of the invention based on the disclosure of WO2016/105,499 where a bile salt is added to the combination of benzyl alcohol and lecithin organogel in lieu of adding an aqueous medium, micelles that would have been relatively spherical may become elongated and worm-like thus permitting superior penetration of the stratum corneum of the epidermis. The worm like formation of the micelles is particularly helpful in accommodating higher molecular weight therapeutic agents.

The inclusion of bile salts thus facilitates the ultradeformability of micelles which, in turn, facilitate passage of low and high molecular weight drugs and other active agents, such as nucleic acids and proteins. These compositions overcome the skin penetration barrier by squeezing themselves along the intercellular sealing lipid thereby following the natural gradient across the stratum corneum. This facilitates a change in membrane composition locally and reversibly when pressed against or attracted to a narrow pore.

Bile salts in combination with lecithin organogel facilitate the factors of micellar stability, enhanced viscosity and viscoelasticity that are critical in transdermal drug delivery. Both thermodynamic and kinetic stability is enhanced by the addition of background electrolytes, such as sodium chloride and sodium citrate. Sodium citrate is strongly ionic, thereby reinforcing the interactions between water molecules and various solutes. These electrolytes can more effectively increase viscosity and viscoelasticity of micelles and screen the repulsion between bile salt anions at a minimal concentration.

In some formulations, formation of micelles is enhanced by milling. The level of enhancement is determined by the pressure and speed at which milling occurs as well as the number of passes through the milling machine. As the number of passes and the pressure is increased, the level of micelle formulation is enhanced as well. In general, increasing the pressure and increasing the speed of milling enhances the level of micelle density.

For the ointment milling machine Dermamill 100 (Blaubrite) marketed by Medisca®, typical speeds include any variation between 1 to 100, where 1 is the slowest speed and 100 is the fastest speed, such as speeds of 1, 5, 10, 15, 20, 25, 30, 35, 40, 45, 50, 55, 60, 65, 70, 75, 80, 85, 90 or 100, or any speed in between. The pressure is selected from 1 to 5, where 1 is the highest pressure and 5 is the lowest pressure. The pressure used can be selected from 1, 2, 3, 4, or 5. The number of passes can also be varied, where a pass is complete when all of the product has passed through the rollers of the machine. Multiple passes, such as 2, 3, 4, 5, 6, 7, 8, 9, 10 or more passes, are contemplated in some embodiments. The speed and pressure can be varied for each pass. For example, a first pass may have a first pressure and first speed, while a second (or subsequent) pass may have a second pressure and second speed, where the second pressure is the same or different from the first pressure and the second speed is the same or different from the first speed. The desired micelle density for particular formulations can be determined empirically by varying the speed, pressure and number of passes.

Alternative ointment milling machines could also be used, and comparable speeds, pressures and numbers of passes are replicated by comparison to the equivalents on the Dermamill 100. Alternatively, micelle densities can be compared microscopically to assure equivalent results to those set forth herein. In some embodiments, the micelle density is at least 20% and in many cases at least 30%, 50%, 70%, 80% or 90% and all levels within this range.

In general, the potential for self-assembly is determined by the mass and composition of the copolymer backbone, the concentration of the polymer chains and the properties of encapsulated or pendant drugs and targeting agents. The contribution of various factors for determining micelle stability of each parameter is presented below:
1) Critical micelle concentration (CMC) or minimum concentration of polymer required for micelles to form.
2) Stability of the micelles to prevent disruption and premature release of the drug cargo before reaching the target site.
   a. Thermodynamic stability is characterized by the CMC.
   b. Drug-core interactions can affect stability.
   c. Interactions between the polymer chains in the corona with each other.
   d. Kinetic stability describes the behavior of the micelle system in aqueous solution.

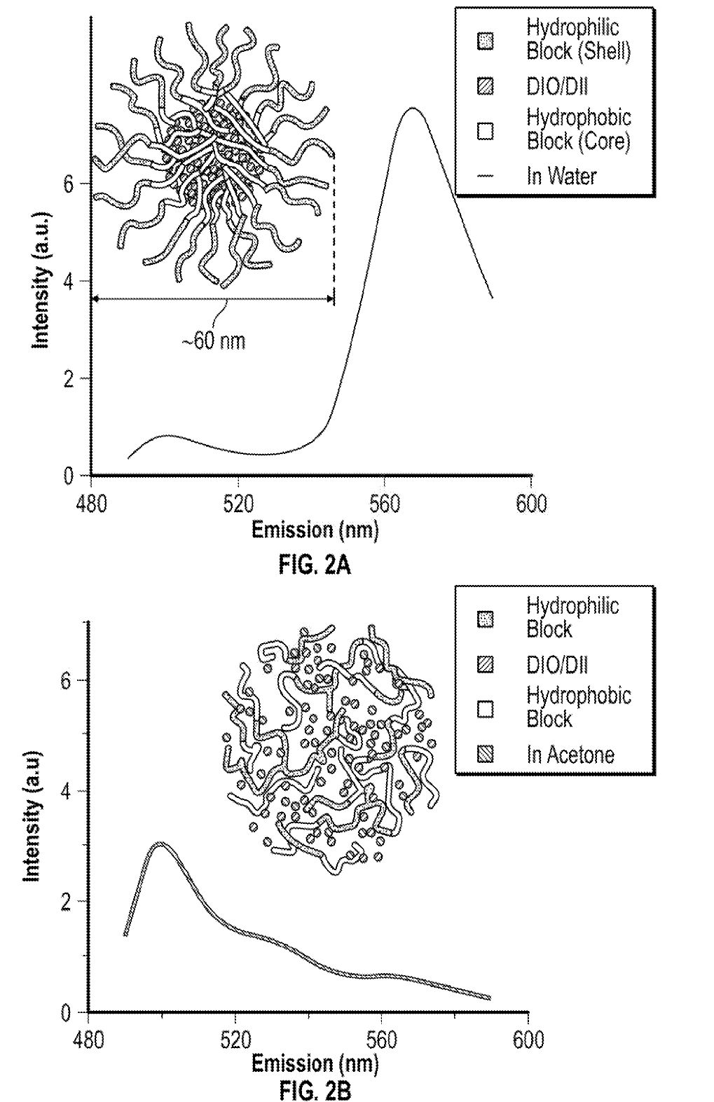

As shown in FIG. 2, the nature of the solvent also has a significant influence on the structure of the micelles obtained.

Composition of the Invention Vehicles

A: Basic CPE Components of One Embodiment

One embodiment comprises approximately equimolar mixtures of a bile salt, a lecithin and a completion component. An “approximate” 1:1:1 ratio is intended to represent a composition of 0.9-1.1:0.9-1.1:0.9-1.1. It has been found that such approximately equimolar mixtures are particularly effective when combined with an electrolyte, a surfactant and benzyl alcohol or an analog thereof. The equimolar mixture comprises 10-75% w/w of the final formulation. In general, when a range of percentages or other parameters is provided herein, the range includes intermediate ranges as well. Thus, the 10-75% w/w presence of the equimolar mixture also includes, for example, 25-75% w/w, 35-75% w/w, 10-70% w/w, 25-50% w/w or 35-45% w/w. Even if specifically not called out, these narrower ranges are included within the scope of the invention.

Bile salts are salts of steroidal acids found in bile. The salts occur in bile in the form of conjugates with taurine or glycine. They are facial amphiphiles and include salts of chenodeoxycholic acid, cholic acid and deoxycholic acid. Salts of these acids with inorganic cations are also members of this class.

As noted above, the inclusion of these bile salts facilitates the ultradeformability of micelles which, in turn, facilitate passage of low and high molecular weight drugs and other active agents such as nucleic acids and proteins. These compositions overcome the skin penetration barrier by squeezing themselves along the intercellular sealing lipid thereby following the natural gradient across the stratum corneum. This facilitates a change in membrane composition locally and reversibly when pressed against or attracted to a narrow pore.

The bile salt may initially be provided in the form of the corresponding acid and by adjustment of pH may be present in the form of the salt, or may be provided as the salt per se.

Lecithin is a biosurfactant and a zwitterionic phospholipid molecule with a head group comprising positively charged choline and a negatively charged phosphate. When a small quantity of completion component, such as water is added to these compounds, the lecithin tends to self-organize into bi-layer membranes and in turn into vesicles or spherical micelles.

The completion component is selected from three alternatives. One alternative is polar and includes water as a polar agent, although other polar agents such as glycerol, ethylene glycol and formamide have been found to possess the capability of transferring an initial non-viscous lecithin solution into a jelly-like state. In a second alternative, the completion component is an organic solvent such as cyclopentane, cyclohexane, cyclooctane, trans-decalin, trans-pinane, n-pentane, n-hexane, n-hexadecane. The third alternative is an amphiphilic ester such as isopropyl palmitate, ethyl laurate, ethyl myristate or isopropyl myristate, or other similar esters.

The ratio of lecithin to completion component is thus approximately 50:50 thus resulting in an organogel. One example is a formulation of soy lecithin in combination with isopropyl palmitate. Other lecithins, such as egg lecithin or synthetic lecithins, are also suitable. Soy lecithin comprised of 96% pure phosphatidylcholine may be used. Various esters of various long chain fatty acids may also be employed in lieu of isopropyl palmitate. Methods for making such lecithin organogels are well known in the art.

This basic formulation also includes one or more electrolytes, one or more surfactants and benzyl alcohol or an analog thereof. The inclusion of an electrolyte results in a viscous and cream-like or gel-like formulation.

Suitable electrolytes are organic or inorganic salts such as sodium or potassium chloride, sodium or potassium citrate and other soluble salts. In preparing these formulations, the amount of electrolyte is added by titration until the mixture becomes transparent, highly viscous and viscoelastic which is noted when the container is inverted. This is helpful for the formation of wormlike micelles that can retain their flexibility and stability and retain their cargo of active agents. The percentage of electrolyte is dependent on the character and amount of the approximately 1:1:1 bile salt:lecithin:completion agent. It is thus determined empirically.

Suitable detergents include Tween® 80 and Span® 80 as well as poloxamers such as Pluronic® and any other surfactant characterized by a combination of hydrophilic and hydrophobic moieties. Poloxamers are triblock copolymers of a central hydrophobic chain of polyoxypropylene flanked by two hydrophilic chains of polyethyleneoxide. Other nonionic surfactants include long chain alcohol and copolymers of hydrophilic and hydrophobic monomers where blocks of hydrophilic and hydrophobic portions are used.

Other examples of surfactants or detergents include polyoxyethylated castor oil derivatives such as HCO-60 surfactant sold by the HallStar Company; nonoxynol; octoxynol; phenylsulfonate; poloxamers such as those sold by BASF as Pluronic® F68, Pluronic® F127, and Pluronic® L62; polyoleates; Rewopal® HVIO, sodium laurate, sodium lauryl sulfate (sodium dodecyl sulfate); sodium oleate; sorbitan dilaurate; sorbitan dioleate; sorbitan monolaurate such as Span® 20 sold by Sigma-Aldrich; sorbitan monooleates; sorbitan trilaurate; sorbitan trioleate; sorbitan monopalmitate such as Span® 40 sold by Sigma-Aldrich; sorbitan stearate such as Span® 85 sold by Sigma-Aldrich; polyethylene glycol nonylphenyl ether such as Synperonic® NP sold by SigmaAldrich; p-(1,1,3,3-tetramethylbutyl)-phenyl ether sold as Triton™ X-100 sold by Sigma-Aldrich; and polysorbates such as polyoxyethylene (20) sorbitan monolaurate sold as Tween® 20, polysorbate 40 (polyoxyethylene (20) sorbitan monopalmitate) sold as Tween® 40, polysorbate 60 (polyoxyethylene (20) sorbitan monostearate) sold as Tween® 60, polysorbate 80 (polyoxyethylene (20) sorbitan monooleate) sold as Tween® 80, and polyoxyethylenesorbitan trioleate sold as Tween® 85 by Sigma-Aldrich. The weight percentage range of surfactant is in the range of 3% w/w-15% w/w, and again includes intermediate percentages such as 5% w/w, 7% w/w, 10% w/w, 12% w/w, and the like.

In some embodiments, the detergent provides suitable handling properties whereby the formulations are gel-like or creams at room temperature. To exert this effect, the detergent, typically a poloxamer, is present at a level of at least 9% w/w, preferably at least 12% w/w in polar formulations. In anhydrous forms of the compositions, the detergent is added in powdered or micronized form to bring the composition to 100% and higher amounts are used. In compositions with polar constituents, the detergent is added as a solution. If smaller amounts of detergent solutions are needed due to high levels of the remaining components, more concentrated solutions of the detergent are employed. Thus, for example, the percent detergent in the solution added may be 5% or 10% to 40% or 20% or 30% and intermediate values depending on the percentages of the other components.

Benzyl alcohol (BA) is exemplified in these formulations; however, benzyl alcohol analogs may also be used. Such analogs include other alcohols with hydrophobic chains especially those wherein an aromatic group is included. Thus other alcohols could also be included or substitute for BA, in particular derivatives of benzyl alcohol which contain substituents on the benzene ring, such as halo, alkyl, amide carboxylates and the like.

The weight percentage of benzyl and/or analog in the final composition is 0.5-20% w/w, and again, intervening percentages such as 1% w/w, 2% w/w, 5% w/w, 7% w/w, 10% w/w, and other intermediate weight percentages are included.

Further, as the skin surface pH is just below 5, increasing the pH of the vehicle will enhance penetration. Adjustment of pH to 7, 8, 9, 10 or 11 is included in the invention.

B: Additional Components of the Vehicles that Enhance Effectiveness

In addition to the basic set of components described above, the penetrant of the invention may include skin penetrating peptides (SPPs or CPPs), which are present at 1% w/w-5% w/w.

The SPPs may function by permeating through the transcellular route passing through hydrophilic keratin-packed corneocytes that are embedded in multiple hydrophobic lipid bilayers. While partitioning into the keratin-rich corneocytes, they form bridges that bind with the filamentous keratin in co-administration as peptide-chaperones without interacting with the guest drugs or degrading the lipid matrix. SPPs may enhance the lipid organization while simultaneously increasing skin electrical conductivity.

The co-administration of SPPs has been postulated to result in a statistically significant increase in percentage of a-helices of keratins, suggesting that SPPs stabilize these structural proteins (keratins). The intra-cellular keratins are stabilized by disulfide bonds, which are tightly packed either in α-chains (α-keratins) or in β-sheet (β-keratins) structures. The high-degree of cross-linking by the disulfide bonds, hydrophobic interactions and hydrogen bonds between the keratin filament structures within the individual corneocytes confer its mechanical stability preventing free drug transport.

It has been reported that the SPPs also utilize the intercellular pathways via small gaps between the corneocytes by disrupting cell-to-cell junctional desmosomes expeditiously, thereby modifying the intercellular spaces from about 30 nm to about 466 nm in as little as 30 minutes from topical administration. This is a transient process that will escort macromolecules across the SC permeation barrier restoring the breaches in about one hour after application.

A number of SPPs are known in the art. It has long been known that the TAT peptide derived from HIV has been able to escort substances through the skin. More recently, WO2007/035474 discloses the peptide TD-1 which has the amino acid sequence ACSSSPSKHCG. A review of such transdermal enhanced peptides (TEPs) which are exemplary of SPPs is published by Ruan, R, et al., Ther. Deliv. (2016) 7:89-100. These include, in addition to TD-1, SPACE, DLP, LP12 and T2. An additional such peptide is disclosed by Gautam, A., et al., Sci. Reports (2016) 6:26278 as IMT-P8 with a sequence RRWRRWNRFNRRRCR.

Additional SPPs are described in WO2016/033314 and U.S. Pat. No. 8,518,871. Suggested mechanisms for SPPs are described by Kumar, S., et al., J. Cont. Rel. (2015) 199:168-178.

Soy lecithin phosphatidylcholine has been revealed to form a noncovalent complex with TD-1, which implies an interaction between TD-1 and the negatively charged cell lipids. Microemulsions consisting of bile salts, lecithin organogel and electrolytes have been used to form supramolecular structure that can increase not only skin permeability but also drug solubility in formulation and drug partitioning into the skin.

In addition to or in lieu of SPPs, the composition described above may be supplemented with agents that are designed to break down the keratin contained in the corneocytes. Keratinolytic agents may disrupt the tertiary structure and hydrogen bonds between individual keratin filaments, reduce disulfide linkages and/or lyse the keratin itself, thereby promoting penetration through intact skin. The administration of keratinolytic agents will release any keratin-bound active drug and enhance bioavailability.

One approach is disruption of the disulfide linkage of the keratin filaments of which the corneocytes are comprised by use of reducing agents such as thioglycolic acid (TGA), dithiothreitol (DTT), and β-mercaptoethanol (β-ME). The amounts included depend on the agent and are effective to reduce the disulfide linkages in the collagen either partially or completely. Urea hydrogen peroxide is believed to disrupt H-bonds. This, too, may be determined empirically, by, for example, determining changes in conformation of the keratin.

Another type of keratinolytic agent is an enzyme, such as Proteinase K@ about 10 mg/mL that can also be employed to degrade the keratin substrate. The optimal pH of keratinolytic activity is around pH 8, while activity is detected in a broad range of pH values between 6 to 11 for serine proteases. Chemical hydrolysis will further compromise the barrier property contributed by the corneocytes but the process is irreversible and concentration-dependent, and the amount to be added is dependent on the degree of lysis required. Typically only small amounts, e.g., 1-5% w/w, need be included.

The simultaneous application of a reducing agent has been demonstrated to have no adverse effect on the keratinolytic enzymes and, in fact, allows the preferential access of the enzymes to the substrate for enhanced proteolytic attack. One keratinolytic product, K4519-500UN (Sigma-Aldrich), is a non-specific serine protease with the capability of degrading insoluble keratin substrates by cleaving non-terminal peptide bonds. Two cooperating enzymes isolated from a keratin-degrading bacterium, Stenotrophomonas sp. strain D-1 disrupt the disulfide bonds while simultaneously degrading the keratin substrate.

In addition to or in lieu of the foregoing components, various miscellaneous permeation enhancers can be employed in suitable amounts. These permeation enhancers include compounds that aid the permeation of macromolecules such as insulin and/or are demonstrated by high throughput electrometric screening to be skin resistance-reduction agents. Such permeation enhancers include binary mixtures of methyl pyrrolidone with dodecyl pyridinium (in a ratio of approximately 1:2) that are identified in this way.

An important class of penetration enhancers are unsaturated and polyunsaturated fatty acids, such as oleic, palmitoleic, alternative unsaturated forms of, for example, myristic acid, lauric acid, undecanoic acid, and the like may also be used. Typically this form of penetration enhancer is supplied as a solution in the benzyl alcohol component. The amounts of total permeation enhancer included are typically in the range of 0.2% w/w to 20% w/w.

Embodiments Modifying CPE

In another embodiment, the foregoing components, the SPPs, reagents that degrade keratin, and permeation enhancers may be used to improve the cell penetrating enhancer (CPE) described in the above-referenced and incorporated herein WO2016/105499, or other known CPEs including but not limited to those described in WO2014/209910 and in US2009/0053290. Briefly, in these formulations, the basic compositions employ lecithin organogels and benzyl alcohol. In some embodiments, a combination of a nonionic surfactant and molar excess of a polar gelling agent or a bile salt and detergent are provided so that the penetration capabilities of the resulting formulation and the effective level of delivery of the active agent are greatly enhanced.

Briefly, WO2016/105499 discloses that the performance of the formulations is further improved by including a nonionic detergent and polar gelling agent or including bile salts and a powdered surfactant. In both aqueous and anhydrous forms of the composition, detergents, typically nonionic detergents are added. In the compositions wherein the formulation is topped off with a polar or aqueous solution containing detergent, the amount of detergent is typically relatively low—e.g., 2%-25% w/w, or 5-15% w/w or 7-12% w/w. However, in compositions that are essentially anhydrous and comprise bile salts are topping-off is by powdered detergent, and relatively higher percentages are usually used—e.g., 20%-60% w/w. The boundaries are not rigid but the above description indicates the general range.

In many embodiments, the pH is in the range of 8.5-11 or 9-11 or 10-11.

The formulations of WO2016/105499, briefly described above, are cell penetration enhancers (CPEs). In the present invention these and other CPEs are supplemented with SPPs and/or keratinolytic agents and/or permeation enhancers. One example of additional known CPEs includes the binary mixtures found to enhance permeation as noted above by high-throughput electrometric screening. In those embodiments where the CPE is represented by this binary mixture, the invention compositions must include either or both an SPP and/or a keratinolytic agent.

The descriptions set forth above with regard to the nature and amounts of additional agents to be included apply to these known CPEs as well. Thus, for example, the percentage content of SPPs set forth above applies here, as well, as do the specifications with regard to permeation enhancers and keratinolytic compounds.

Components Included in Formulations Comprising the Vehicles of the Invention

A: Active Agents

The active agents in the formulations are varied, and the appropriate choice of formulations will depend on the nature of the active agent in that the molecular weight and polar or non-polar character of the active agent may favor particular embodiments of the vehicles described herein. In general, active agents that are macromolecules are favored by the inclusion of the skin penetrating peptides as well as the intracellular acting components such as reducing agents for disulfide bonds and proteolytic agents that dissolve keratin. Lower molecular weight components may benefit as well. Typical active agents are either therapeutic (including nutritional) or diagnostic compounds that are desired to be delivered beneath the skin or through the nails locally or are destined to enter the synthetic circulation. The active ingredient could be simply a nutrient, an antibiotic, an anesthetic, a protein such as insulin, an oligonucleotide, an antibody, a molecule selected from the vast array of pharmaceuticals currently available or in development, and the like. The invention does not lie in the nature of the active ingredient, but rather in the nature of the penetrant vehicle itself.

The percentage of active agent in the formulation will depend upon the concentration required to be delivered in order to have a useful effect on treating the disorder. In general, the active ingredient may be present in the formulation in an amount as low as 0.01% w/w up to about 50% w/w. Typical concentrations include 0.25% w/w, 1% w/w, 5% w/w, 10% w/w, 20% w/w and 30% w/w. Since the required percentage of active ingredient is highly variable depending on the active agent and depending on the frequency of administration, as well as the time allotted for administration for each application, the level of active ingredient may be varied over a wide range, and is limited only by the necessity for including in the formulation aids in penetration of the skin by the active ingredient.

The formulations of the invention may include only one active agent or a combination of active agents. In the present application, “active agent” or “active ingredient” refers to a compound or drug that is active against the factors or agents that result in the desired therapeutic or other localized systemic effect.

In general, in the present application, “a,” “an,” “one,” and the like should be interpreted to mean one or more than one unless it is clear from the context that only a single referent is intended. For example, “an active ingredient” may refer to one or more such active ingredients, and “a permeation enhancer” includes mixtures of these.

B: Miscellaneous Optional Components

One or more anti-oxidants may be included, such as vitamin C, vitamin E, proanthocyanidin and α-lipoic acid typically in concentrations of 0.1%-2.5% w/w.

Various excipients may be added. Excipients that may be used in some embodiments include β- and γ-cyclodextrin complexes, hydroxypropyl methylcellulose (such as Carbopol® 934), or other thickening agents.

Also included are components present essentially for aesthetic reasons such as menthol, fragrances, coloring agents and other components that do not alter the penetration capability of the formulations but rather are added for alternative reasons. Preservatives such as paraben may also be included.

Another class of compounds that may be included and is often helpful is one or more antiseptics. Cetyltrimethyl ammoniumbromide, for example, is included in the exemplified composition.

In some applications, it is desirable to adjust the pH of the formulation to assist in permeation or to adjust the nature of the active agent and/or of the target compounds in the subject. In some instances, the pH is adjusted to a level of pH 9-11 or 10-11 which can be done by providing appropriate buffers or simply adjusting the pH with base.

As noted above, in some embodiments other additives are included such as a thickener, a dispersing agent or a preservative. An example of a suitable thickener is hydroxypropylcellulose, which is generally available in grades from viscosities of from about 5 cps to about 25,000 cps such as about 1500 cps. The concentration of hydroxypropylcellulose may range from about 1% w/w to about 2% w/w of the composition. Other thickening agents are known in the art and can be used in place of, or in addition to, hydroxypropylcellulose. One example is Durasoft® PK-SG (polyglycerol-4-laurate) at 1-3% w/w preferably 2% w/w to prevent separation and act as an emulsifier/thickener. Another example is absorbent phyllosilicate clays, such as bentonite. An example of a suitable dispersing agent is glycerin. Glycerin is typically included at a concentration from about 5% w/w to about 25% w/w of the composition. A preservative may be included at a concentration effective to inhibit microbial growth, ultraviolet light and/or oxygen-induced breakdown of composition components, and the like. When a preservative is included, it may range in concentration from about 0.01% w/w to about 1.5% w/w of the composition.

Preparation

The formulations of the invention may be prepared in a number of ways. Typically, the components of the formulation are simply mixed together in the required amounts. However, it is also desirable in some instances to, for example, carry out dissolution of an active ingredient and then add a separate preparation containing the components aiding the delivery of the active ingredients in the form of a carrier. The concentrations of these components in the carrier, then, will be somewhat higher than the concentrations required in the final formulation.

Alternatively some subset of these components can first be mixed and then “topped off” with the remaining components either simultaneously or sequentially. The precise manner of preparing the formulation will depend on the choice of active ingredients and the percentages of the remaining components that are desirable with respect to that active ingredient.

Treatments Administered Subsequent to Effecting Skin Penetration

The primary function of the epidermis is to generate a tough, protective sheath, the SC, by virtue of its formidable permeability barrier, and thus in the course of topical and transdermal drug delivery, this permeation barrier must be compromised. Effective transdermal delivery requires disruption of the permeation barrier resulting in transient essential fatty acid deficiency with special reference to the elimination of linoleic acid. Enhancing drug delivery across intact skin results in barrier disruption that will predispose the skin to the vulnerability of increased transepidermal water loss (TEWL), invasion of toxins and inflammatory processes

Thus a post-procedural repair process reversing the iatrogenic vulnerability of percutaneous delivery is desirable. One embodiment is application of linoleic acid available from many natural products, such as sun flower seeds, evening primrose oil, safflower oil, refined fish oil, kukui nut oil in a formulation that comprises linoleic acid in concentrations of from about 0.5% to about 5% (w/w). Such a replacement formulation may also include 1% carbomer hydrogel with from about 0.3% to about 10% liposomal ursolic acid to result in ceramide synthesis. Return of TEWL to normal signifies successful repair.

In another embodiment, calcium salts such as calcium carbonate, calcium chloride and calcium gluconate, in concentrations of from about 0.1% to about 5% may be applied to drive keratinocytes into differentiation and stimulate the cells to synthesize additional ceramides.

In still another embodiment, to reverse the degradation facilitated by the keratinolytic corrosion of the corneocyte surfaces, serine-proteinase inhibitor PMSF may be employed, as well as Cu²⁺ and Mn²⁺ and Ca²⁺, Mg²⁺, Zn²⁺, ethanol and isopropyl alcohol.

The following examples are offered to illustrate but not to limit the invention.

### EXAMPLE 1

An exemplary formulation includes:
1. Cetyltrimethyl ammoniumbromide (from about 2.0% w/w to about 10.0% w/w) (surfactant and antiseptic*)
2. Sodium cholate: Lecithin (96% pure): Isopropyl myristate (equi-molar 1:1:1) (from about 10% w/w to about 40.0% w/w)
3. Sodium citrate (titrate to transparency/incr. viscosity of #2) (electrolyte)
4. Thioglycolic acid (from about 1.0% w/w to about 10.0% w/w) (reducing agent) [may be substituted by Urea Hydrogen peroxide @ about 20.0% w/w]
5. Benzyl alcohol (from about 2.0% w/w to about 20.0% w/w)
6. Cis-Palmitoleic acid (from about 0.4% w/w to about 6% w/w supplied as a 20% w/w-30% w/w solution in the benzyl alcohol—permeation enhancer)
7. Methyl pyrrolidone (0.4%)/Dodecyl pyridinium (1.1%) (from about 0.5% w/w to about 5.0% w/w) (permeation enhancer)
8. Pluronic® to top off (detergent)
* Also enhances insulin penetration of cells

### EXAMPLE 2

The composition of Example 1 is combined with one or more of:
1. TD-1: ACSSSPSKHCG (SPP) as needed
2. Thioglycolic Acid (TGA) (from about 2.0% w/w to about 7.0% w/w concentration)
3. Proteinase K (from about 5 mg/mL to about 15 mg/mL)

### EXAMPLE 3

### Physical Parameters

Steady and dynamic rheological experiments on the invention formulation are performed on a Rheometrics RDA-III strain-controlled rheometer. Frequency spectra are conducted in the linear viscoelastic regime of the samples, as determined from dynamic strain sweep measurements.

Small angle neutron scattering (SANS) measurements are made on the NG-7 (30 m) beamline at NIST in Gaithersburg, MD. Neutrons with a wavelength of 6 A are selected. Samples are prepared with deuterated cyclohexane and studied in 1 mm quartz cells at 25° C. The scattering spectra are corrected and placed on an absolute scale using calibration standards provided by the National Institute of Standards and Technology (NIST).

For dilute solutions of non-interacting scatters, the SANS intensity can be modeled purely in terms of the form factor P(q) of the scatterers. In this study, we considered form factor models for three different micellar shapes; ellipsoids, rigid cylinders and flexible cylinders. The models were implemented using software modules supplied by NIST.

These methods of testing are based on studies at the University of Maryland.

### EXAMPLE 4

### Clinical Studies of Chemical Permeation Enhancement

Clinical trials are performed on the invention formulations applied twice daily for 45 days. Several dermatologists and plastic surgeons will observe the patients. Documentation of objective results is performed with the microrelief technique. The technique relies upon the application of a polyvinylsiloxane impression material to the skin. Upon drying, the film is removed and either sputter coated with a conducting metal for visualization utilizing a scanning electron microscope and/or a high power stereomicroscope and photography. Each scale division equals 0.5 mm.

Adjacent sites which remain untreated are used as a control.

The specimens are processed for histological evaluation. Standard dehydrating and paraffin embedding procedures are used. The specimens are stained with H & E and alcian blue to visualize the collagen and proteoglycan components of the extracellular matrix.

The treated skin shows significant differences as compared with the control. The dermis in the treated specimen shows a greater abundance of collagen with characteristics that depict a more recently deposited fibrous network. The epithelial layer is much thicker, well organized and reflects a greater cellular metabolic activity. Such results confirm effective and expeditious percutaneous absorption of the active agent.

These methods are based on studies at the Keck School of Medicine, University of Southern California.

### EXAMPLE 5

### Percutaneous Penetration

A skin model from University of Illinois School of Medicine utilizes normal, human-derived epidermal keratinocytes and normal, human-derived dermal fibroblasts, which have been cultured to create a multi-layered, highly differentiated model of human dermis and epidermis in a three-dimensional tissue construct, which is metabolically and mitotically active. The tissues are cultured on specially prepared cell culture inserts using serum-free medium. Ultrastructurally, this model closely parallels human skin, thus providing a useful in vivo means to assess percutaneous absorption or permeability. The model has an in vivo-like lipid profile with in vivo-like ceramides present. Furthermore, this model reproduces many of the barrier function properties of normal human skin and has been determined to be a useful substrate for percutaneous absorption, transdermal drug delivery and other studies related to the barrier function of the human.

Donor solution (PBS) containing four different concentrations (0.25 g/ml, 0.5 g/ml, 1 g/ml, and 2 g/ml) of the invention composition or control base is prepared. Neutral red (0.001%) is added to give a red tinge to the donor solution.

The donor solution is then added to the center core of the permeation device containing the skin tissue and the whole assembly is then placed into the wells of a 6 well plate containing 3 ml of PBS. At definite intervals, the assembly is moved to a fresh well containing 3 ml. of PBS. After incubation, PBS from the 6 wells were collected in separate tubes, labeled and stored in −70° C. for further processing. After 120 hours of incubation will confirm that all skin tissue samples in this study are viable at the end of the study period.

### EXAMPLE 6

### Transepidermal Water Loss Measurements

The rate of transepidermal water loss (TEWL) (g/h/m2) is reflective of the skin's barrier function. In a method based on materials from BioScreen Testing Services, Inc., a TEWL probe utilizing the DermaLab® Evaporimeter System (Cortex Technology, Hadsund Denmark) is used to take three baseline measurements on both the left and right volar forearms. The template demarcated test sites are then tape stripped (Duct tape, 3M™, St. Paul, Minn.). Following tape stripping, TEWL measurements are again taken at each tape stripped site. Increased TEWL indicates a disruption of the permeation barrier of the SC following the topical application of the chemical permeation enhancement compositions.

### EXAMPLE 7

### Collagen Message Levels

A real time polymerase chain reaction method from University of Illinois School of Medicine is used to determine collagen message levels in the human dermal fibroblast cell lines exposed to the penetration sample compound (at concentrations of 0.25 mg/ml) and base control (at 0.25 mg/ml concentrations). Cells incubated in media alone serve as negative controls.

Absolute quantities of collagen are determined in the fibroblasts using a real time polymerase chain reaction analysis. cDNA is prepared from the fibroblasts using a RETROscript® real time polymerase chain reaction kit.

These analyses show that exposure to the penetration sample compound induces the expression of collagen in human dermal fibroblasts within 30 minutes. Similar changes are not observed at 30 minutes when the base was applied to fibroblast cultures.

### EXAMPLE 8

### Electrometric Analysis of Permeability of Human Epidermis

Skin conductivity is generally a good measure of its permeability to polar solutes. Transepidermal current is mediated by the movement of charge carrying ions and is thus related to the permeability of these ions. For screening purposes, the skin possessing higher electrical conductivity exhibits higher permeability to polar solutes. Therefore, monitoring electrical conductivity of skin exposed to various permeation enhancing formulations will identify the most efficient formulations in increasing skin permeability as performed using a method developed at University of California, Santa Barbara.

### EXAMPLE 9

### Elemental Analysis

A proton-induced X-ray spectrographic technique developed by University of Illinois School of Medicine is used for the non-destructive, simultaneous elemental analysis of solid, liquid or aerosol filter samples. To determine if the sample has penetrated through the epidermal layer, the PBS samples collected after incubation are subjected to elemental analysis (Table: Elemental Analysis).

Samples are analyzed by proton induced X-ray analyzer, which measures 74 elements in one run with special interest in two elements, copper (Cu) and iron (Fe).

Results of the proton induced X-ray analysis will confirm that (1) the penetrant sample dose penetrated the epidermis (2) within 30 minutes of application. Thus the compound is available to the deeper layers, especially dermal fibroblasts within 30 minutes of its application to the epidermal surface.

### EXAMPLE 10

### High Performance Liquid Chromatography Analysis

The concentration of insulin in the receiver well at different time intervals is measured using a HPLC system. A 40:60 (v/v) mixture of acetonitrile and water is the mobile phase. Flow rate is 1.0 mL/min. and the eluent is monitored at 276 nm linearity for HPLC analysis is observed in the concentration range of 0.01-12.5 IU/ml (R²>0.99).

This is a technique used to separate, identify and quantify each component in a mixture. Each component in the sample interacts slightly differently with the absorbent material, causing different flow rates for the different components and leading to the separation of the components as they flow out the column. It is a mass transfer process involving adsorption.

### EXAMPLE 11

### Permeability Coefficient and Enhancement Factor Calculations

In this study, the amount of drug permeated is calculated as the total amount of drug permeated through skin during a time period of 48 hours. The lag time is calculated as the x-intercept of the steady state portion of the permeation profiles (cumulative insulin permeated, IU/cm²) plotted against the time (hr) profiles.

The following steady-state equation is used to calculate permeability of the skin:

Amount of drug permeated=Aₘ*C₀*Kₚ* t

where, Aₘ is the exposure area of the skin sample (0.64 cm²), C₀ is the initial concentration in the well in mm, Kₚ is the permeability of the membrane and t is time in hours. The permeability is give in terms of the diffusion coefficient (Dₘ), the partition coefficient (Kₘ), and the thickness of the skin sample (L):

Kₚ₌DₘKₘ/L.

1. A vehicle for effecting transdermal penetration of an active ingredient wherein said vehicle comprises:
i) an approximately 1:1:1 equimolar mixture of bile salt:lecithin:completion component;
ii) one or more electrolytes sufficient to impart viscosity and viscoelasticity to the vehicle;
iii) one or more surfactants; and
iv) benzyl alcohol or an analog thereof;
wherein the completion component is a polar liquid, a non-polar liquid or an amphiphilic substance.

2. The vehicle of claim 1 which further comprises a keratinolytic agent effective to reduce thiol linkages, disrupt hydrogen bonding and/or effect keratin lysis.

3. The vehicle of claim 1 which further comprises at least one skin penetrating peptide (SPP).

4. The vehicle of claim 2 which further comprises at least one skin penetrating peptide (SPP).

5. The vehicle of claim 1 which further comprises a permeation enhancer.

6. The vehicle of claim 2 which further comprises a permeation enhancer.

7. The vehicle of claim 3 which further comprises a permeation enhancer.

8. The vehicle of claim 4 which further comprises a permeation enhancer.

9. The vehicle of claim 1 which comprises micelles.

10. A vehicle for effecting transdermal penetration of an active ingredient wherein said vehicle comprises:
i) lecithin organogel;
ii) benzyl alcohol or an analog thereof; and
iii) a keratinolytic agent effective to reduce thiol linkages, disrupt hydrogen bonding and/or effect keratin lysis.

11. The vehicle of claim 10 which further comprises at least one skin penetrating peptide (SPP).

12. The vehicle of claim 10 which further comprises a permeation enhancer.

13. The vehicle of claim 11 which further comprises a permeation enhancer.

14. The vehicle of claim 10 which comprises micelles.

15. A vehicle for effecting transdermal penetration of an active ingredient wherein said vehicle comprises:
i) a lecithin organogel;
ii) benzyl alcohol or an analog thereof; and
iii) at least one SPP.

16. The vehicle of claim 15 which further comprises a permeation enhancer.

17. The vehicle of claim 15 which comprises micelles.

18. A composition for delivery of an active agent which comprises an effective amount of said agent in combination with the vehicle of claim 1.

19. A method to deliver an active agent to a subject which method comprises applying the composition of claim 18 to the skin of said subject.

20. (canceled)

21. A composition for delivery of an active agent which comprises an effective amount of said agent in combination with the vehicle of claim 10.

22. A composition for delivery of an active agent which comprises an effective amount of said agent in combination with the vehicle of claim 15.

23. A method to deliver an active agent to a subject which method comprises applying the composition of claim 21 to the skin of said subject.

24. A method to deliver an active agent to a subject which method comprises applying the composition of claim 22 to the skin of said subject.
